(19) United States
(12) Patent Application Publication
(10) Pub. No.: US 2018/0103898 A1
(43) Pub. Date: Apr. 19, 2018

(54) METHOD AND SYSTEM FOR VISUALIZING MECHANICAL VENTILATION INFORMATION

(71) Applicant: General Electric Company, Schenectady, NY (US)

(72) Inventors: Scott William Robinson, Bayside, WI (US); Andreas Tzanetakis, Helsinki (FI); Nathaniel Brazy, Madison, WI (US); Bryan Powell, Longmont, CO (US)

(21) Appl. No.: 15/843,166

(22) Filed: Dec. 15, 2017

Related U.S. Application Data
(63) Continuation of application No. 13/198,535, filed on Aug. 4, 2011.

(51) Int. Cl. A61B 5/00; A61M 16/00

(52) U.S. Cl. CPC A61B 5/4848; A61M 16/024; A61M 16/0051; A61B 5/14542; G06F 19/3406; A61M 2205/505

(57) ABSTRACT

A medical system having a ventilator is provided. The medical system includes a memory for storing one or more patient and ventilation parameters. A processor is programmed to organize the one or more patient parameters into at least one of past patient and ventilation parameters, present patient and ventilation parameters, or future patient and ventilation parameters. A viewer displays at least one of the past patient or ventilation parameters, the present patient or ventilation parameters, or the future patient or ventilation parameters. The past patient and ventilation parameters, the present patient and ventilation parameters, and the future patient and ventilation parameters are selectable on the viewer.

### CROSS-REFERENCE TO RELATED APPLICATIONS

This application is a continuation of U.S. patent application Ser. No. 13/198,535, which was filed on 4 Aug. 2011, and the entire disclosure of which is incorporated herein by reference.

### BACKGROUND OF THE INVENTION

The subject matter disclosed herein relates generally to respiratory care systems, and more particularly, to mandatory mechanical ventilation systems.

When patients are medically unable to breathe on their own, mechanical or forced ventilators can sustain life by providing requisite pulmonary gas exchanges for the patients. For example, conventional ventilators typically include electronic and pneumatic systems that control the pressure, flow rates, and/or volume of gases delivered to, and extracted from, patients needing medical respiratory assistance. Such control systems often include numerous user controls, such as knobs, dials, switches, and the like, for interfacing with treating clinicians, who support the patient's breathing by adjusting the pressure, flow rates, and/or volume of the patient's pulmonary gas exchanges, particularly as the condition and/or status of the patient changes. These parameter adjustments are challenging to control accurately, particularly using these conventional systems.

With respect to ventilation, this is a complex process of delivering oxygen to, and removing carbon dioxide from, alveoli within patients' lungs. Thus, conventional ventilators, particularly controlled mechanical ventilation (CMV) systems, include inputs that allow operating clinicians to select and use several modes of ventilation, either individually and/or in various combinations, using different ventilator setting controls. These mechanical ventilators have become increasingly sophisticated and complex, due in part to enhanced understandings of lung pathophysiology. Accordingly, many conventional ventilators are microprocessor-based and equipped with sensors that monitor patient pressure, flow rates, and/or volumes of gases, and then drive automated responses in response thereto. However, as these ventilators become more complicated and provide more options, the number and risk of potentially dangerous clinical decisions increases as well. Thus, clinicians often operate expensive, sophisticated machines, yet few follow clear, concise, and/or consistent guidelines for maximal use thereof. For example, numeric information may be collected into tables and spreadsheets. Accordingly, clinicians may be required to search through pages of tabular data searching for critical numeric indicators of patient status. As a result, setting, monitoring, and interpreting ventilation parameters may be reduced to empirical judgment, resulting in less than optimal treatment. For example, tracking past patient data along with present patient data and future extrapolated data may be time consuming.

Thus, the overall effectiveness of assisted ventilation ultimately depends on mechanical, technical, and physiological factors, with the clinician-ventilator-patient interface playing an important role. For example, clinicians often need to observe and control several factors to optimize the volume of air that is appropriate given the particular patient. However, it is often difficult for clinicians to observe and control these several factors at the same time.

### SUMMARY OF THE INVENTION

In one embodiment, a medical system having a ventilator is provided. The medical system includes a memory for storing one or more patient and ventilation parameters. A processor is programmed to organize the one or more patient parameters into at least one of past patient and ventilation parameters, present patient and ventilation parameters, or future patient and ventilation parameters. A viewer displays at least one of the past patient or ventilation parameters, the present patient or ventilation parameters, or the future patient or ventilation parameters. The past patient and ventilation parameters, the present patient and ventilation parameters, and the future patient and ventilation parameters are selectable on the viewer.

In another embodiment, a method for presenting ventilator data is provided. The method includes storing one or more patient and ventilation parameters and organizing the one or more patient parameters into at least one of past patient and ventilation parameters, present patient and ventilation parameters, or future patient and ventilation parameters. At least one of the past patient or ventilation parameters, the present patient or ventilation parameters, or the future patient or ventilation parameters are displayed on a viewer. The past patient and ventilation parameters, the present patient and ventilation parameters, and the future patient and ventilation parameters are selectable on the viewer.

In another embodiment, a non-transitory computer readable storage medium for displaying ventilator information using a processor is provided. The non-transitory computer readable storage medium includes instructions to command the processor to store one or more patient and ventilation parameters. The one or more patient parameters are organized into at least one of past patient and ventilation parameters, present patient and ventilation parameters, or future patient and ventilation parameters. At least one of the past patient or ventilation parameters, the present patient or ventilation parameters, or the future patient or ventilation parameters are displayed on a viewer. The past patient and ventilation parameters, the present patient and ventilation parameters, and the future patient and ventilation parameters are selectable on the viewer.

### BRIEF DESCRIPTION OF THE DRAWINGS

FIG. 1 is a simplified block diagram of a medical system in accordance with various embodiments.

FIG. 2 illustrates screenshots of a display formed in accordance with an embodiment.

FIG. 3 is an exemplary view of a past screen displayed in accordance with an embodiment.

FIG. 4 is an exemplary view of a present screen displayed in accordance with an embodiment.

FIG. 5 is an exemplary view of another present screen displayed in accordance with an embodiment.

FIG. 6 is an exemplary view of another present screen displayed in accordance with an embodiment.

FIG. 7 is an exemplary view of a future screen displayed in accordance with an embodiment.

FIG. 8 is an exemplary view of another future screen displayed in accordance with an embodiment.

FIG. 9 is an exemplary view of another future screen displayed in accordance with an embodiment.

FIG. 10 is a flowchart illustrating a method for displaying data from a ventilator.

### DETAILED DESCRIPTION OF THE INVENTION

The foregoing summary, as well as the following detailed description of certain embodiments, will be better understood when read in conjunction with the appended drawings. The figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

The various embodiments provide a user interface for a patient ventilator. In particular, various embodiments provide a time based viewer providing a user interface for a patient ventilator. Specifically, the viewer displays a visual representation of patient and/or ventilation parameters. For example, the patient and ventilation parameters may be related to a patient's breathing ability while on a ventilator and corresponding ventilator settings. At least one technical effect of various embodiments is the ability to monitor the patient and ventilation parameters in the past and present, and provide clinical decision support for future patient treatment.

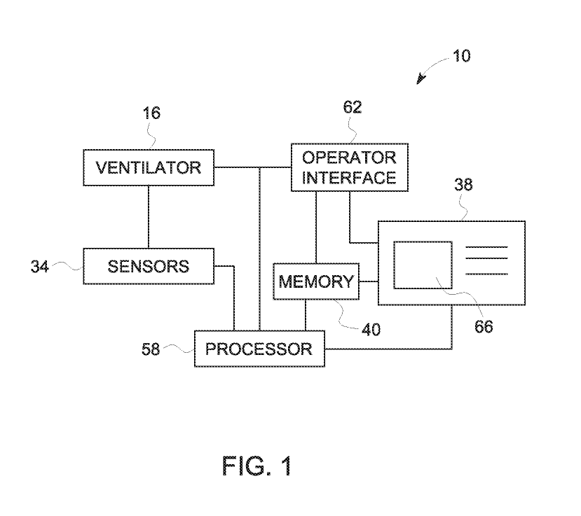

The viewer may be implemented in a patient monitoring system providing mechanical ventilation. For example, the viewer may be implemented in a medical system 10 as illustrated in FIG. 1. The medical system 10 in various embodiments provides for mechanically ventilating a patient (not shown). The medical system 10 also provides for visualization of the patient and ventilation (or ventilator) parameters for use in controlling a ventilator 16 based on displayed compliance data in combination with measurement data from one or more sensors 34. The ventilator may be controlled via an operator interface 62 by a clinician viewing the visualized patient and ventilation parameter data, which may be multiple data types displayed concurrently on a monitor 38 to allow a user to view a balance between at least some of the patient and ventilation parameters. For example, a user may view a plurality of past, present, and/or future patient and ventilation parameters. A processor 58, for example, a processing subsystem of the medical system 10 may process received measurements from the sensors 34 and other compliance information as described herein to update the monitor 38 with the patient and ventilation parameter data. The medical system 10 may include a memory 40 for storing data related to the patient and ventilation parameters. The processor 58 may be programmed to organize data related to the patient and ventilation parameters.

The ventilator 16 further can receive inputs from the sensors 34 associated with the patient (e.g., coupled to the patient) and/or the ventilator 16 at the processor 58 for subsequent processing thereof, and which can be displayed on the monitor 38. Representative data received from the sensors 34 can include, for example, inspiratory time (T_{I}), expiratory time (T_{E}), natural exhalation time (T_{EXH}), respiratory rates (f), I:E ratios, positive end expiratory pressure (PEEP), fractional inspired oxygen (F_{I}O₂), fractional expired oxygen (F_{E}O₂), breathing gas flow (F), tidal volumes (V_{T}), temperatures (T), airway pressures (P_{aw}), arterial blood oxygen saturation levels (SₐO₂), blood pressure information (BP), pulse rates (PR), pulse oximetry levels (SₚO₂), exhaled CO₂ levels (F_{ET}CO₂), concentration of inspired inhalation anesthetic agent (C_{I} agent), concentration of expired inhalation anesthetic agent (C_{E} agent), arterial blood oxygen partial pressure (PₐO₂), arterial carbon dioxide partial pressure (PₐCO₂), and the like.

The components are functionally depicted for illustration, wherein various components thereof can also be integrated and/or separated, as needed and/or desired. Other functional components, for example, one or more power supplies for the medical system 10 and/or ventilator 16 may be provided.

Various embodiments provide a viewer 66 that is a user interface tool for the medical system 10, and specifically for controlling operation of the ventilator 16. For example, the viewer 66 enables a user, such as a clinician, to balance and evaluate the patient and ventilation parameters in the past and present and facilitate a determination of future settings or parameters. Thus, the viewer 66 allows control of one more ventilation parameters or settings based on displayed information, which may be related in part to patient physiology. Accordingly, the various embodiments allow clinicians to view and control patient and/or ventilation parameters throughout the respiratory cycle of the patient and enables ventilation control or treatments to be individually controlled (e.g., optimized) for patients subject to controlled mechanical ventilation (CMV).

The ventilator 16 may be used to measure different conditions or parameters, for example, inspiratory time (Ti). Ti is the amount of time, measured in seconds, set on the ventilator 16 by the clinician, lasting from the beginning of the inspiration of the patient to the beginning of the expiration of the patient. The ventilator 16 may also be configured to measure other parameters, for example, expiratory time (T_{E}), I:E ratios (e.g. the ratios between T_{I} and T_{E}), natural exhalation time (T_{EXH}), positive end expiratory pressure (PEEP), tidal volume (V_{T}), respiratory rate (f), and/or any other parameters generally measured by a ventilator.

The viewer 66 may be used to adjust the settings of the ventilator 16 to balance patient and ventilation parameters. In particular, using the visualizations provided by various embodiments, the degree to which an ideal balance between the patient and ventilation parameters has been achieved may be visually observed and determined, thereby allowing a user, such as a clinician to adjust the settings of the ventilator 16, such as to change the initial settings of ventilation parameters.

In various embodiments, “ventilation settings” or “ventilator settings” generally refers to parameters that determine or define how ventilation is provided to the patient. These settings are parameters selected by a clinician. In various embodiments, patient parameters are measured values that are data generally referring to the reading acquired from the sensors 34 (shown in FIG. 1 and which may be connected to the patient directly or indirectly) that represent the status of the patient. It should be noted that some quantities can be both a setting and a measurement, and some quantities may be either depending on the current ventilation mode. It should be noted that compliance ranges are also generally settings (or alarm settings). Thus, patient parameters generally refer to measured data and ventilation parameters generally refers to ventilation settings.

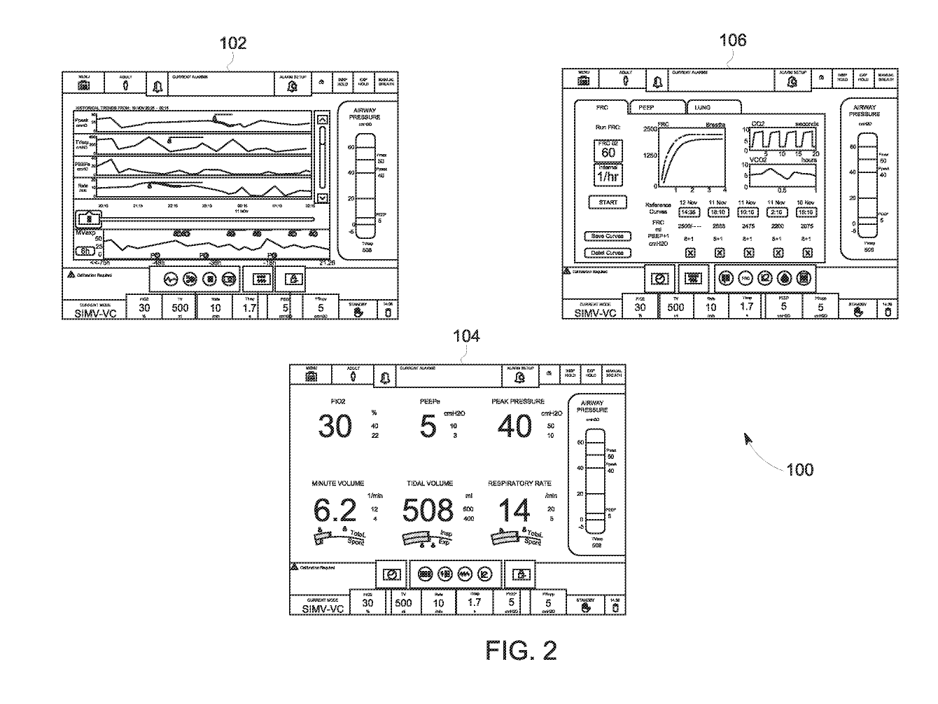

FIG. 2 illustrates screenshots of a display 100 formed in accordance with an embodiment. In one embodiment, the display 100 is embodied as the viewer 66 shown in FIG. 1. The display 100 may be, for example, a touch screen display. Alternatively, the display 100 may be coupled to a user input device, for example, a keyboard, a mouse, or the like. FIG. 2 illustrates, generally, a past screen 102 showing a first feature set representing past patient and ventilation parameters (e.g. settings, measurements, etc.), a present screen 104 showing a second feature set representing present patient and ventilation parameters, and a future screen 106 showing a third feature set representing possible future ventilation settings (which may be based on predicting or forecasting measured values), and are referred to herein as future patient and ventilation parameters. The future patient and ventilation parameters may provide, for example, clinical decision support for future actions. An operator may toggle between the past screen 102, the present screen 104, and the future screen 106 using a touch screen display. Alternatively, the operator may toggle between the screens 102, 104, and 106 using a keyboard, a knob, or the like.

The display 100 provides time based navigation of the patient and ventilation parameters by toggling between the past screen 102, the present screen 104, and the future screen 106. The display 100 organizes information and interaction with respect to the patient and ventilation parameters according to the paradigm of time to provide natural intuitive guidance regarding where the patient and ventilation parameters may be accessed, reviewed, and analyzed.

The display 100 provides a system for structuring the review and control of patient and ventilation parameters in the domains of the past, present and future. The past screen 102 organizes and generally displays patient and ventilation parameters that have already taken place and the tools used to analyze and communicate recorded information and events related to the patient and ventilation parameters. The past screen 102 may be provided, for example, as described in the co-pending US application entitled “METHOD AND SYSTEM FOR VISUALIZING MECHANICAL VENTILATION INFORMATION”, having attorney docket number 242157 GD (553-1704) and which is commonly owned. The present screen 104 organizes and generally displays patient and ventilation parameters that are currently taking place and the tools used to analyze and communicate what is transpiring with respect to the patient and ventilation parameters. The future screen 106 organizes and generally prepares for the collection of data related to the patient and ventilation parameters and the tools used to configure the data collection. The future screen 106 also generally enables an operator to plan for the future behavior of the ventilator and/or ancillary devices. For example, the future screen 106 provides a tool to assist a clinician in assessing what changes to ventilation settings might result in a particular desired patient outcome (or close to that outcome). Thus, the future screen 106 in various embodiments provides a means to evaluate possible changes in ventilator settings to determine what changes may be optimal for the patient, which is not necessarily a predictive measure.

The display 100 includes at least one of the past screen 102, the present screen 104, and/or the future screen 106 so that a user can ascertain or predict the location of and/or more quickly navigate to desired data and/or to a desired system feature. The screens 102, 104, and 106 may be displayed separately, simultaneously, concurrently, and on the same or different screens.

In one embodiment, the past screen 102 may display information related to trends of measures of patient and ventilation parameters (e.g. respiration rate, heart rate, blood pressure level, volume of breath, or the like, as well as ventilator settings), alarms that have occurred in the past (e.g. measured patient and ventilation setting parameters, device problems, or the like), events that have occurred in the past (e.g. changes in device settings, therapeutic processes, or the like), or recordings of system and patient status that have occurred in the past (e.g. screen shots, periods of data recordings, or the like). The present screen 104 may display information related to current measured patient and ventilation parameters (e.g. respiration rate, heart rate, blood pressure level, volume of breath, waveforms, or the like, as well as ventilator settings), current alarms (e.g. measured patient and ventilation setting parameters, device problems, or the like), device settings, and/or user selectable variations on the layout of measured patient values and corresponding graphic representations. The future screen 106 may display information related to available therapeutic procedures or protocols that can be invoked or scheduled for the future, analysis tools to determine and schedule future device settings, and/or patient status forecasting tools.

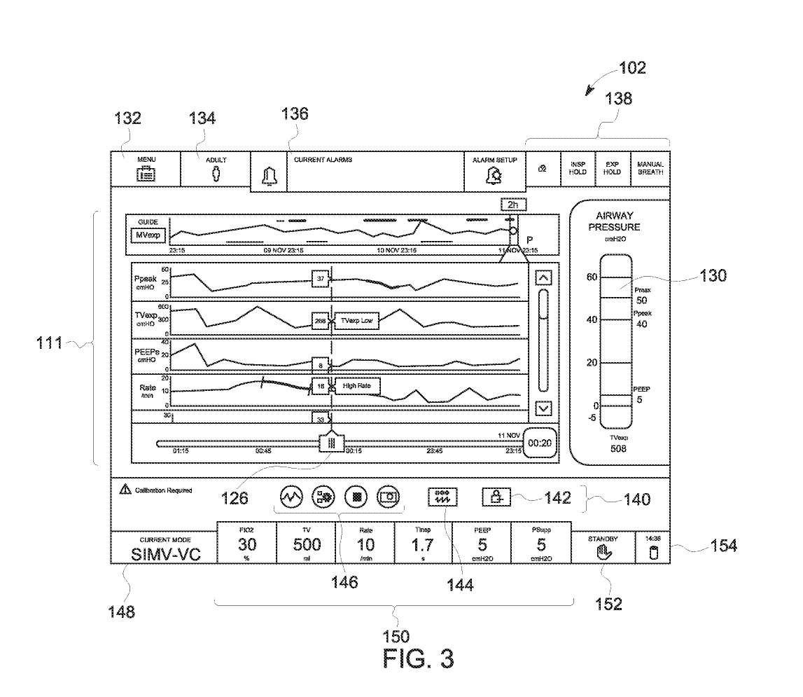

FIG. 3 is an exemplary view of a past screen 102 including a patient and ventilation parameter display 111. The patient and ventilation parameter display 111 displays a visualization of past patient and ventilation parameters. The patient and ventilation parameter display 111 may include a master timeline and local timelines, as described in copending application entitled “METHOD AND SYSTEM FOR VISUALIZING VENTILATION INFORMATION”, having attorney docket no. 242157 (553-1704), and being commonly owned.

In the illustrated embodiment, a scale 130 is displayed with the patient and ventilation parameter display 111. The scale 130 represents an airway pressure of the patient. In one embodiment, the scale 130 may be toggled on and off by an operator. The scale 130 may be provided, for example, as described in copending application Ser. No. 13/112,870, entitled “METHOD AND SYSTEM FOR VISUALIZING VENTILATION INFORMATION”, and commonly owned. The scale 130 generally shows present patient information regardless of whether past and/or future data is displayed on the rest of the screen.

A menu button 132 is provided for selecting a menu, for example, a menu screen or drop down menu that enables the operator to access, for example, system settings, as well as to configure and run different procedures (which may be in the present or future). A patient button 134 is provided for selecting a type of patient, for example, adult, child, or infant. Various operating parameters may be updated based on the type of patient. Additionally, various compliance ranges may updated based on the type of patient. An alert screen button 136 is activated to display current alert notifications. The alert notifications may also be accompanied by visual and/or audible alarms. In one embodiment, the alert screen button 136 may activate a drop down screen that displays the most recent alerts. Function buttons 138 are provided to instruct the ventilator to perform various functions. It should be noted that although the various buttons are shown as user selectable soft keys (e.g. virtual buttons displayed on the screen), the buttons may be any type of hard or soft button, key, etc. Additionally, instead or in addition to buttons, any type of user interaction control or input, whether virtual or physical may be provided.

View buttons 140 are provided to change an appearance of the past screen 102, view a present screen, or view a future screen. For example, by selecting a future screen button 142, a future screen is displayed illustrating future patient and ventilation parameters. By selecting a present screen button 144, a present screen is displayed illustrating present patient and ventilation parameters. Additionally, multiple past screen buttons 146 are provided to change a view or format of the past screen 102. The operator may toggle between the past screen 102, a present screen, and/or a future screen, by selecting the corresponding view buttons 140. Additionally, the operator may toggle between the various views and formats of the past screen 102 by selecting the corresponding view buttons 140. For example, the past screen 102 may be displayed in various graphical or numerical formats.

A mode button 148 may be selected to change and/or update an operating mode of the ventilator. Parameter buttons 150 display desired compliance levels for various parameters. The parameter buttons 150 may be selected to alter the corresponding compliance levels. A standby button 152 may be selected to pause the operation of the ventilator. Operation of the ventilator may be paused during various patient treatments, for system calibration, or the like. A battery display 154 indicates a battery level of at least one of the monitor 38 and/or the ventilator 16 (both shown in FIG. 1).

It should be noted that although the embodiments are described with respect to various functional buttons, not all of the functional buttons are required to practice the embodiments described herein. Additionally, various other functional buttons may be included on screens, for example, the past screen 102.

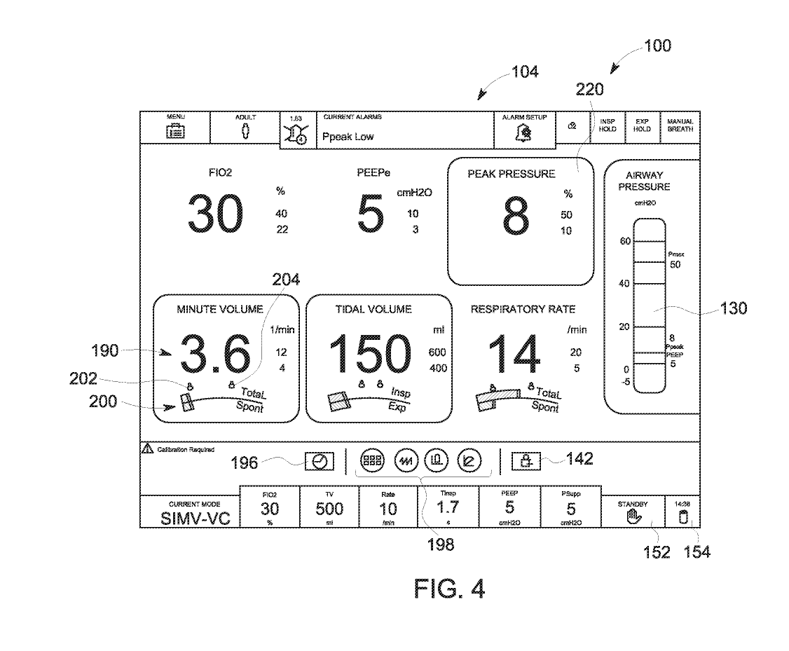

FIG. 4 is an exemplary view of a present screen 104 displayed in accordance with an embodiment on the display 100. The present screen 104 displays real time values 190 of various patient and ventilation parameters. The parameters displayed may be selected by the operator. In the illustrated embodiment, the present screen 104 is configured to display the values 190 of six parameters. Alternatively, the present screen 104 may be configured to display the values 190 of more or less than six parameters. The values 190 update on the present screen 104 in real time. The values 190 may be displayed with compliance ranges 200 having a low value 202 and a high value 204. If one of the parameters falls outside of the corresponding compliance range 200 an alert notification may be activated. The operator may adjust either one of the low value 202 or high value 204 accordingly.

In the illustrated embodiment, the present screen 104 displays alert notifications 220 on the display 100. The alert notifications 220 are illustrated as boxes around the patient and ventilation parameter values 190. The alert notifications 220 may be color coded based on a severity of the alert. For example, if a parameter value 190 has fallen slightly outside of the compliance range 200, the alert notification 220 may be a color such as yellow (indicating a medium priority alert). If a parameter value 190 has severely fallen outside of the compliance range 200, the alert notification may be a color such as red. Additional visual and/or audio alerts may be triggered if a parameter value 190 falls outside of the compliance range 200. In one embodiment, the alert notification may include a symbol and/or an audio alert.

The future screen button 142 is provided along with a past screen button 196 and multiple present screen buttons 198. The operator may toggle between the past screen 102, the present screen 104, and the future screen 106 by selecting the future screen button 142, the past screen button 196, and the present screen buttons 198. An operator may toggle between various present screens 104 having different formats by selecting one of the present screen buttons 198.

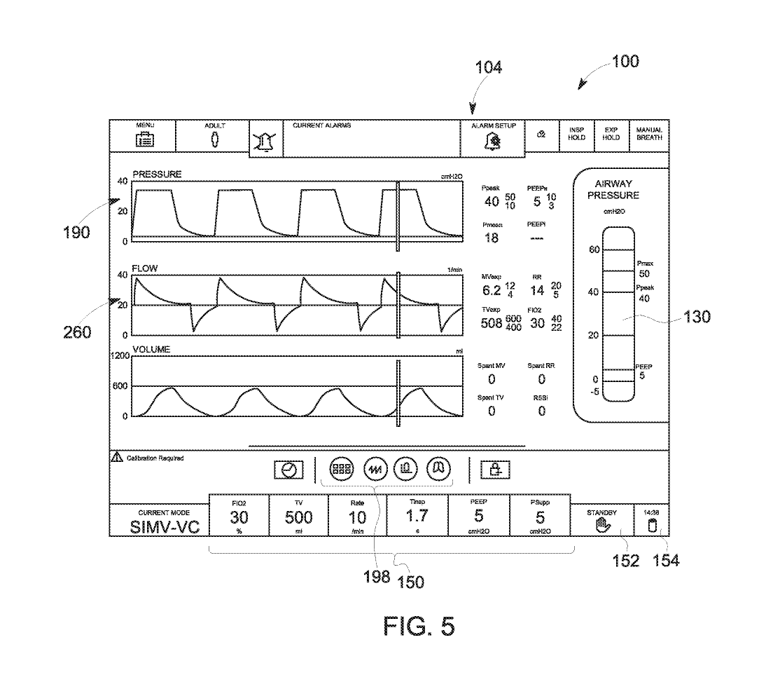

FIG. 5 is another exemplary view of the present screen 104 on the display 100. FIG. 5 illustrates the parameter values 190 as waveforms 260. An operator may choose the format of the present screen illustrated in FIG. 5 by selecting one of the present screen format buttons 198. The operator may toggle between the view illustrated in FIG. 5 and other views using the present screen buttons 198. The waveforms 260 enable the operator to view a snapshot of the data of the parameter values 190.

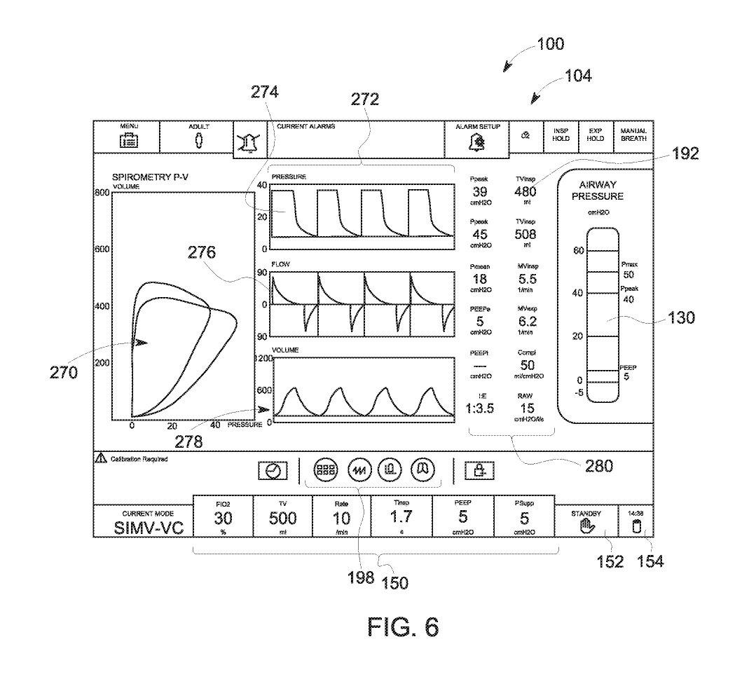

FIG. 6 is another exemplary view of the present screen 104 on the display 100. FIG. 6 illustrates the parameter values 190 as a spirometry chart 270. An operator may choose the format of the present screen illustrated in FIG. 6 by selecting one of present screen buttons 198. The operator may toggle between the view illustrated in FIG. 6 and other views using the present screen buttons 198. The spirometry chart 270 enables the operator to view the data of the parameter values 190 in a desired format.

Additionally, the waveforms 272 are displayed with the spirometry chart 270 to illustrate various ventilation parameters. For example, in the illustrated embodiment, a pressure graph 274 of air from the ventilator, a flow graph 276 of air from the ventilator, and a volume graph 278 of air from the ventilator are displayed. In other embodiments, other ventilator and patient parameters may be displayed. In the illustrated embodiment, various parameter values 190 are displayed in a chart 280.

The present screens 104 shown in FIGS. 4-6 can provide various means with which to review present patient and ventilation parameters. The various present screens 104 enable an operator to review the present patient and ventilation parameters in a format that is best understood or desired by the operator. For example, the operator may prefer to view patient data in a waveform format or one or two configurable spirometry chart formats. Another operator may change the view to display a format preferred by that operator.

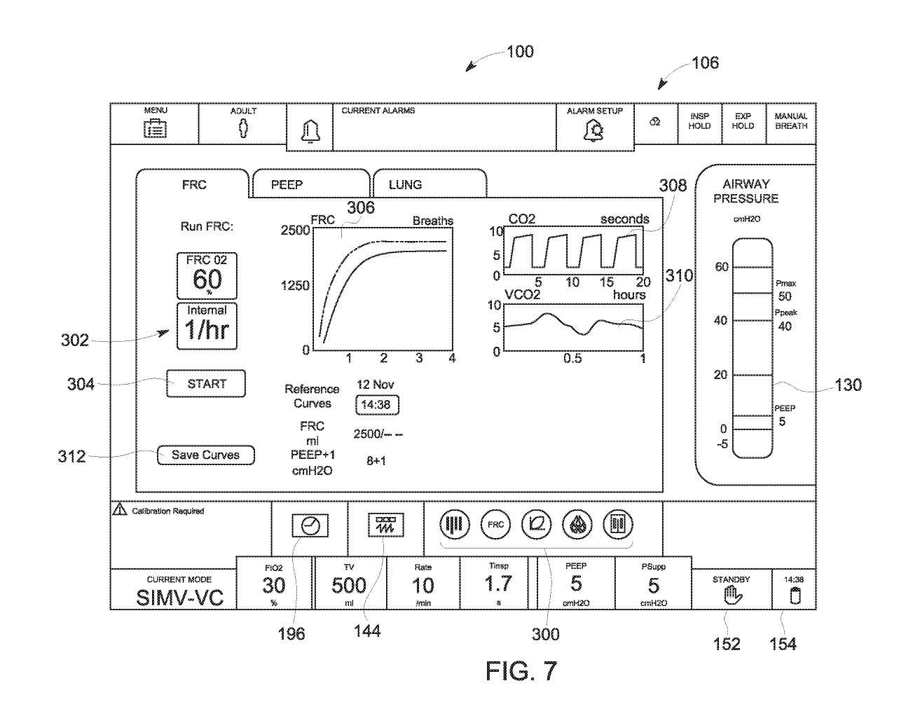

FIG. 7 is an exemplary view of a future screen 106 displayed on the display 100. The future screen 106 is provided to chart a path of treatment for the patient. In one embodiment, the future screen 106 may display proposed ventilator settings and future patient parameter values that may be, for example, extrapolated from the past and present patient parameters. For example, the future patient parameter may be extrapolated with any suitable extrapolation algorithm or the like. The present screen button 144 is provided along with the past screen button 196 and multiple future screen buttons 300. The operator may toggle between the past screen 102, the present screen 104, and the future screen 106 by selecting the present screen button 144, the past screen button 196, and the future screen buttons 300. An operator may toggle between various future screens 104 having different formats by selecting one of the future screen buttons 300.

In the illustrated embodiment, a functional residual capacity (FRC) screen 302 is displayed. The FRC represents a volume of air present in the patient's lungs at the end of passive expiration. A start button 304 enables the operator to run an FRC test on the patient. The results of the test are displayed in an FRC curve 306. Additionally, a waveform 308 and a curve 310 are provided to illustrate the volume of carbon dioxide in the patient's lungs. The FRC curve 306, the waveform 308, and the curve 310 may be saved by selecting the save button 312. The tools on the FRC screen 302 allow a clinician to estimate what affect changes in ventilator settings may have on the patient's FRC (providing some of the future functionality of the various embodiments).

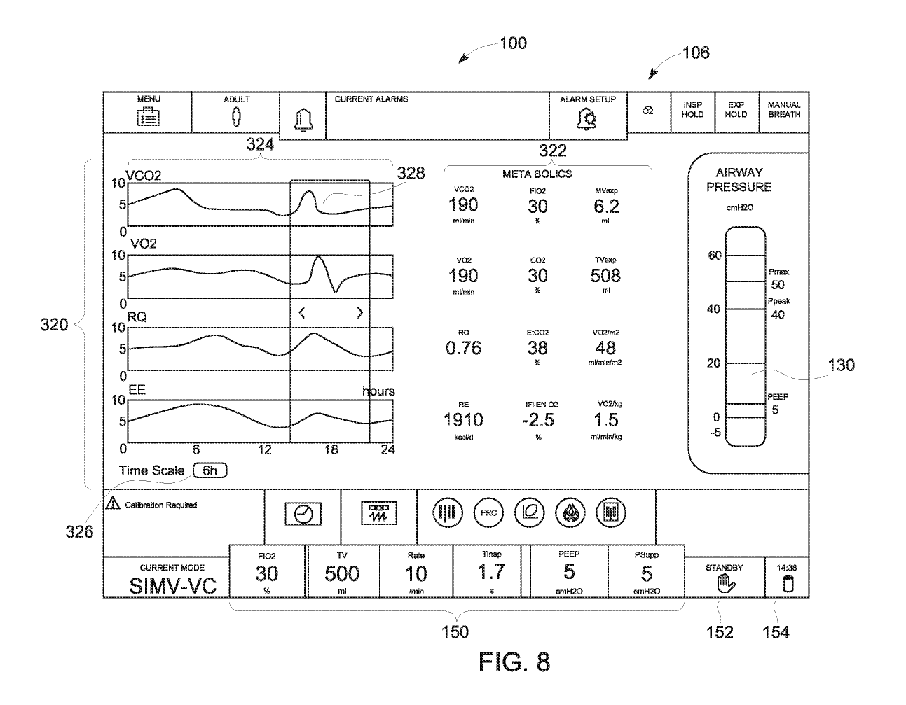

FIG. 8 is another exemplary view of the future screen 106 displayed on the display 100. FIG. 8 illustrates metabolic values 320 for the patient. The metabolic values 320 may be used to chart a path of treatment for the patient to achieve the values 320. The metabolic values 320 are displayed in a numerical chart 322. Additionally, at least some of the metabolic values 320 are displayed in graphs 324. In one embodiment, the graphs 324 may include an adjustable time scale 326. A reference marker 328 may be provided to select data points on the graphs 324 similar to the graphs described above. The numerical chart 322 may be updated based on the data points selected with the reference marker 328. In one embodiment, the metabolic values 320 illustrated in the graph display include measured data for the past twenty-four hours. A clinician may use this information to identify one or more time periods (as indicated on the graphs) over which to calculate, for example, the average metabolic measurements, which are shown in the numerical chart 322.

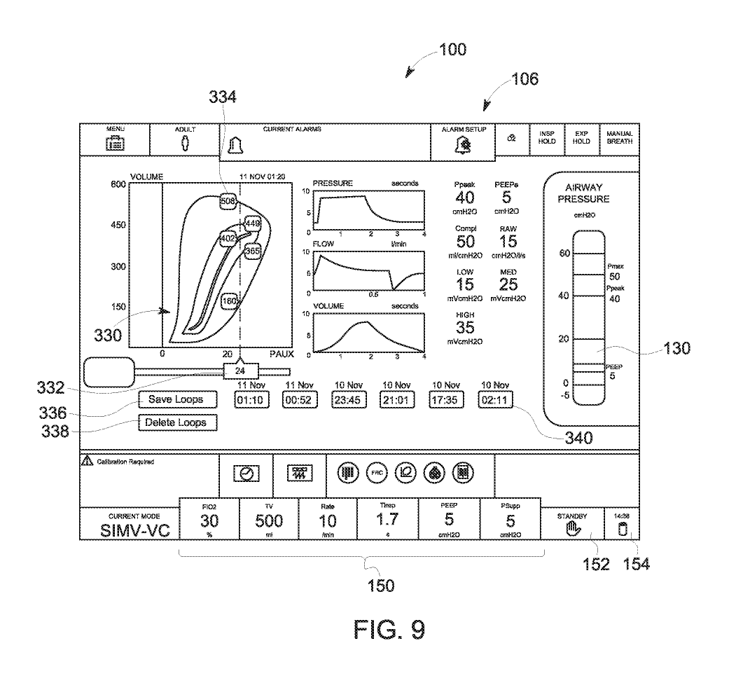

FIG. 9 is another exemplary view of the future screen 106 displayed on the display 100. FIG. 9 displays extrapolated patient parameter values in a spirometry chart 330. The spirometry chart 330 may be used to chart a path of treatment for the patient. The chart 330 may include a reference marker 332 to select various data points 334 on the spirometry chart 330. The loops created on the spirometry chart 330 may be saved with a save button 336 and/or deleted with a delete button 338. Saved loops may be later accessed by selecting a saved loop button 340.

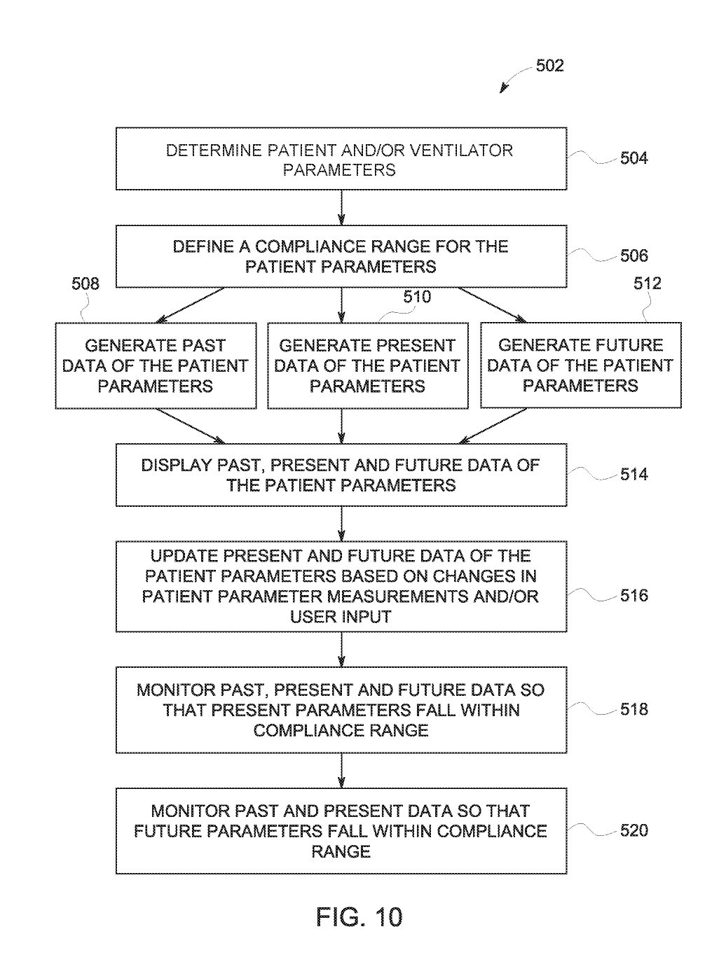

FIG. 10 is a flowchart illustrating a method 502 for displaying data from a ventilator. In one embodiment, instructions may be stored on a tangible non-transitory computer readable storage medium to command a processor, for example, the processor 58 (shown in FIG. 1) to perform the method 502. At 504, patient parameters are determined. For example, the operator may select various patient parameters to monitor with the display. Additionally, the operator may select or set various ventilation parameters to monitor. At 506, a compliance range is selected for each parameter. The compliance range defines a high and low value for the parameter. If a value of the parameter falls below the low value or exceeds the high value, an alert notification is triggered. The compliance range may be manually selected by the operator. Alternatively, predetermined compliance ranges may be selected.

At 508, a first feature set related to each of the parameters is generated. The first feature set is organized and displayed, at 514, for example, on the past screen 102 and represents past patient and ventilation parameters. The first feature set may be displayed in various different formats based on a desired format of the operator. The first feature set may be displayed with a reference line that enables the operator to select specific data points within the first feature set. At 510, a second feature set related to each of the parameters is generated. The second feature set is organized and displayed, at 514, for example, on the present screen 104 and represents present patient parameters. The second feature set may be displayed in various different formats based on a desired format of the operator. At 512, a third feature set related to each of the parameters is generated. The third feature set may be extrapolated from the first feature set and the second feature set. The third feature set is provided, for example, to chart a path of patient treatment. The third feature set is displayed, at 514, for example, on the future screen 106 and represents future patient parameters. The future patient parameters may be generated by aggregating the past patient parameters and the present patient parameters to provide clinical decision support and treatment planning. The third feature set may be displayed in various different formats based on a desired format of the operator.

At 516, the first feature set, the second feature set, and the third feature set are updated. The feature sets may be updated in real time as the present parameters are measured. The feature sets may be updated based on changes in one of the patient parameters. Alternatively, the feature sets may be updated based on an operator input. At 518, each of the first, second, and third feature sets are monitored and/or adjusted so that the present patient parameters fall within the compliance range. In one embodiment, an operator may make adjustments to the ventilator settings and/or provide medical treatment to the patient based on the first, second, and third feature sets so that the present parameters fall within the compliance range. At 520, the first and second feature sets are monitored to help determine the future progress of the patient. In one embodiment, an operator may make adjustments to the ventilator settings and/or provide medical treatment to the patient based on the first and second feature sets so that path of treatment set forth in the third feature set is achieved.

In various embodiments, the method 502 includes storing one or more sensor measurements related to the patient and ventilation parameters. A visualization of the past patient and ventilation parameters, the present patient and ventilation parameter, and the future patient and ventilation parameters is then displayed. An operator is able to select at least one of the past patient and ventilation parameters, the present patient and ventilation parameters, and/or the future patient and ventilation parameters. The past patient and ventilation parameters of the patient may be displayed as a timeline of the past patient and ventilation parameters. In one embodiment, at least one of the past patient and ventilation parameters, the present patient and ventilation parameters, or the future patient and ventilation parameters are displayed in more than one format. An operator is able to toggle between the formats. A scale representing an airway pressure of the patient may be displayed with at least one the past patient and ventilation parameters, the present patient and ventilation parameters, or the future patient and ventilation parameters. In one embodiment, the future patient and ventilation parameters are utilized to provide clinical decision support and treatment planning. The past patient and ventilation parameters and the present patient and ventilation parameters are then monitored to achieve the path of patient treatment. At least one of the past patient and ventilation parameters or the present patient and ventilation parameters also may be monitored so that present patient parameters of the patient fall within the compliance range. In one embodiment, at least one of the past patient and ventilation parameters, the present patient and ventilation parameters, or the future patient and ventilation parameters is displayed in at least one or a graphical or numerical format. An alert may be displayed if one of the present patient and ventilation parameters falls outside of a compliance range. In various embodiments, the past patient and ventilation parameters, the present patient and ventilation parameters and the future patient and ventilation parameters are displayed on a touch screen display. An operator is able to toggle between the past patient and ventilation parameters, the present patient and ventilation parameters, and the future patient and ventilation parameters by operating the touch screen display.

Thus, various embodiments provide for visualization of ventilator related information or data, such as the display of visualizations of past, present, and future patient and ventilation parameters. The visualizations may be numerical, graphical or a combination thereof. Additionally, the various visualizations and displayed indicators may be modified as desired or needed, for example, based on user preferences or system settings.

Exemplary embodiments of a medical system with a ventilator are described above in detail. The components illustrated are not limited to the specific embodiments described herein, but rather, components of the system may be utilized independently and separately from other components described herein. For example, the medical system components described above may also be used in combination with other medical systems, such as medical imaging or diagnostic systems.

It should be noted that the various embodiments, for example, the modules described herein, may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive, optical disk drive, solid state disk drive (e.g., flash drive of flash RAM) and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software. Further, the software may be in the form of a collection of separate programs, a program module within a larger program or a portion of a program module or a non-transitory computer readable medium. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for presenting ventilator data, the method comprising:
measuring patient and ventilation parameters from a ventilator and sensors, wherein the patient and ventilation parameters include metabolic values;
storing the metabolic values;
receiving a selection of past metabolic values;
generating future metabolic values based on the selection of past metabolic values; and
displaying the future metabolic values.

2. The method of claim 1, further comprising receiving a selection of metabolic values within a positionable reference marker, wherein the reference marker represents an average metabolic measurement for a set time period.

3. The method of claim 1, wherein the future metabolic values comprise a resting energy expenditure (RE) value.

4. The method of claim 1, wherein the generating step includes charting a path of treatment of the future metabolic values based on the past metabolic values comprising volumetric oxygen consumption (VO₂) and volumetric carbon dioxide production (VCO₂).

5. The method of claim 4, wherein the charting operation is further based on a respiratory quotient (RQ) or an energy expenditure (EE).

6. The method of claim 1, further comprising displaying a scale that represents a real time airway pressure of the patient.

7. The method of claim 1, further comprising displaying parameter buttons representing compliance levels for the future metabolic values.

8. A medical system having a ventilator, the medical system comprising:
a ventilator and at least one sensor for measuring patient and ventilation parameters, wherein the patient and ventilation parameters include metabolic values, wherein the metabolic values are stored in a memory;
a viewer having a user interface, wherein the viewer is configured to receive a selection of past metabolic values;
a processor operably coupled to the ventilator and the at least one sensor, the processor programmed to generate future metabolic values based on the selection of past metabolic values; and
the viewer is configured to display the future metabolic values.

9. The medical system of claim 8, wherein the viewer is configured to receive a selection of metabolic values within a positionable reference marker, wherein the reference marker represents an average metabolic measurement for a set time period.

10. The medical system of claim 8, wherein the future metabolic values comprise a resting energy expenditure (RE) value.

11. The medical system of claim 8, wherein the processor is programmed to chart a path of treatment of the future metabolic values based on the past metabolic values comprising volumetric oxygen consumption (VO₂) and volumetric carbon dioxide production (VCO₂).

12. The medical system of claim 11, wherein the processor is programmed to chart a respiratory quotient (RQ) or an energy expenditure (EE).

13. The medical system of claim 8, wherein the viewer is configured to display a scale that represents a real time airway pressure of the patient.

14. The medical system of claim 8, wherein the viewer is configured to display parameter buttons representing compliance levels for the future metabolic values.

15. A non-transitory computer readable storage medium for displaying ventilator information using a processor, the non-transitory computer readable storage medium including instructions to command the processor to:
measure patient and ventilation parameters from a ventilator and sensors, wherein the patient and ventilation parameters include metabolic values;
store the metabolic values;
receive a selection of past metabolic values;
generate the future metabolic values based on the selection of past metabolic values; and
display the future metabolic values.

16. The non-transitory computer readable storage medium of claim 15, wherein the instructions command the processor to receive a selection of the metabolic values within a positionable reference marker, wherein the reference marker represents an average metabolic measurement for a set time period.

17. The non-transitory computer readable storage medium of claim 15, wherein the future metabolic values comprise a resting energy expenditure (RE) value.

18. The non-transitory computer readable storage medium of claim 16, wherein the instructions command the processor to chart a path of treatment of the future metabolic values based on the past metabolic values comprising volumetric oxygen consumption (VO₂) and volumetric carbon dioxide production (VCO₂).

19. The non-transitory computer readable storage medium of claim 15, wherein the instructions command the processor to display a respiratory quotient (RQ) or an energy expenditure (EE).

20. The non-transitory computer readable storage medium of claim 15, wherein the instructions command the processor to display parameter buttons representing compliance levels for the future metabolic values.
